(19) United States
(12) Patent Application Publication
(10) Pub. No.: US 2020/0093782 A1
(43) Pub. Date: Mar. 26, 2020

(54) ARIANTS OF 2-[6-(4-CHLOROPHENOXY)HEXYL]-OXIRANE-2-CARBOXYLIC ACID FOR USE IN THE TREATMENT, PREVENTION AND/OR AMELIORATION OF BRAIN DISEASES

(71) Applicant: Meta-IQ ApS, Aarhus C (DK)

(72) Inventors: Josephus Dirk Nieland, Aalborg Ø (DK); Jette Goller Kloth Nieland, Aarhus C (DK)

(21) Appl. No.: 16/472,509

(22) PCT Filed: Dec. 27, 2017
(86) PCT No.: PCT/EP2017/084632

§ 371 Date: Jun. 21, 2019

(30) Foreign Application Priority Data
Dec. 29, 2016 (EP) 16207324.1

(51) Int. Cl. A61K 31/336; A61P 25/00

(52) U.S. Cl. CPC A61K 31/336; A61P 25/00

(57) ABSTRACT

The present invention relates to 2-[6-(4-chlorophenoxy)hexyl]-oxirane-2-carboxylic acid for use in the treatment, prevention and/or amelioration of disorders caused by delipidation of neural tissue. Specific aspects relate to certain administration or dosing patterns, routes of administration. In one embodiment of the present invention is the invention Multiple Sclerosis (MS) or an MS associated disease.

### FIELD OF THE INVENTION

The present invention relates to 2-[6-(4-chlorophenoxy)hexyl]-oxirane-2-carboxylic acid for use in the treatment, prevention and/or amelioration of disorders caused by delipidation of neural tissue. Specific aspects relate to certain administration or dosing patterns, as well as routes of administration. In one embodiment of the present invention is the invention Multiple Sclerosis (MS) or an MS associated disease, and also brain diseases like depression.

### BACKGROUND OF THE INVENTION

2-[6-(4-chlorophenoxy)hexyl]-oxirane-2-carboxylic also known as Etomoxir has been extensively tested in clinical studies for diabetes and cardiovascular diseases. Etomoxir is an irreversible inhibitor of carnitine palmitoyltransferase-1 (CPT-1) on the outside of the outer mitochondrial membrane. This prevents the formation of acyl carnitines, a step that is necessary for the transport of fatty acyl chains from the cytosol into the intermembrane space of the mitochondria, and there with prevents the transport of carnitine acyl chains into the mitochondria. This transport step is necessary to make acyl chains available for beta oxidation and production of ATP from fatty acid oxidation.

It has been found that a number of severe mental and neurological diseases are caused by or are related to delipidation of neural tissue and in particular delipidation of myelin sheets. It has suggested that these severe diseases could be effectively treated or prevented by blocking of the enzyme Carnitine-Palmitoyl-Transferase-1 (CPT-I), but so far has no drugs entered clinical trials or have otherwise been tested on human patients.

Examples of mental disorders include depression and impairment of recent and remote memory (loss of short and long term memory). Many neurological disorders result in impairment of the control of the body, e.g. as seen in Multiple sclerosis (MS). Ideally, treatments for mental and neurological disorders should be aimed at curing the disease. To date, this ideal has not been reached. Known treatments for the disorders are not cures, but merely palliatives, aimed at reducing symptoms to provide the patient with an acceptable quality of life or slowing down the progression of the disease. For example, the drugs that presently available for the treatment of depression take about 4 weeks before they start working and they work in maximally 50% of the patients. In memory impairment that can be induced by different diseases, like stress, there is no treatment. For multiple sclerosis the drugs on the market, can only slow down the progression of the disease. These therapies, which are relatively non-specific, have significant side-effects.

Consequently, the problem underlying the present invention resides in providing an improved therapy for the treatment of mental and neurological diseases, including MS. It has been known for quite some time that in Multiple Sclerosis (MS) lipid metabolism is affected. One of the earlier articles that looked at lipid levels in not only MS patients but also bi-polar disorders and schizophrenia found a reduction in these lipid levels specifically 16.1 and 18.1 lipids are affected. In these studies, it can be seen that in specifically in bipolar disorder and Multiple Sclerosis the poly, mono and saturated fatty acids are downregulated. MS affects approx. 400.000 people in the US and more than 2.5 million worldwide. In the US, prevalence estimates are 90 in 100.000 population; in Europe, the prevalence is between 50-100 per 100.000. The usual onset is between 20-40 years but can be in all age ranges.

There are several different forms of MS. Relapsing-remitting MS (RRMS) is the most common form of the disease, where symptoms appear for several days to weeks, after which they usually resolve spontaneously. After tissue damage accumulates over many years, patients often enter the secondary progressive stage of MS (SPMS), where pre-existing neurologic deficits gradually worsen over time. Relapses can be seen during the early stages of SPMS, but are uncommon as the disease further progresses. About 15% of patients have gradually worsening manifestations from the onset without clinical relapses, which defines primary progressive MS (PPMS). Patients with PPMS tend to be older, have fewer abnormalities on brain MRI, and generally respond less effectively to standard MS therapies. Progressive relapsing MS is defined as gradual neurologic worsening from the onset with subsequent superimposed relapses. Progressive relapsing MS (and possibly a proportion of PPMS) is suspected to represent a variant of SPMS, where the initial relapses were unrecognized, forgotten, or clinically silent.

The functions of lipids are several in the CNS, one function is being part of the function of the myelin sheet, but also other functions like trafficking and function of proteins in the myelin sheet and transport of proteins from oligodendrocytes to the myelin sheet are medicated by lipids.

PCT/EP2009/057983 discloses CPT-I inhibitors for use in treating and/or preventing disorders caused by delipidation of neural tissue. The administration can, amongst many options, be in the form of a tablet. The document does not disclose any human data, tests in oral administration, specific isomer variants of etomoxir, or specific dosages indicating how a human individual should be treated.

Importantly has etomoxir never been found to be effective in crossing the blood-brain-barrier (BBB) after oral administration, and it has consequently been impossible to design experiments for clinical testing of etomoxir in an oral administration form on human patients.

### SUMMARY OF THE INVENTION

In its broadest aspect, the present invention relates to etomoxir with the chemical formula 2-[6-(4-chlorophenoxy)hexyl]-oxirane-2-carboxylic acid, for use in the treatment, prevention and/or amelioration of a brain disease linked to lipid metabolism in a human individual.

An embodiment relates to the specific diseases or disorders selected from the group consisting of MS, MS associated disease, depression, Alzheimer's, and Parkinson's ALS.

A preferred aspect of the present invention relates to R(+)-etomoxir ethyl ester with the chemical formula R(+)-2-[6-(4-chlorophenoxy)hexyl]-oxirane-2-carboxylic acid ethyl ester, for use in the treatment or amelioration of Multiple Sclerosis (MS) or an MS associated disease.

A preferred embodiment of the present invention relates to R(+)-etomoxir ethyl ester with the chemical formula R(+)-2-[6-(4-chlorophenoxy)hexyl]-oxirane-2-carboxylic acid ethyl ester, for use in the treatment or amelioration of Multiple Sclerosis (MS) or an MS associated disease, wherein the R(+)-etomoxir ethyl ester is administered according to an administration pattern comprising: administration of 80 mg/day to a person in need thereof.

Another preferred embodiment of the present invention relates to etomoxir for use according to the present invention, wherein the 80 mg/day R(+)-etomoxir ethyl ester is administered at two times 40 mg/day to a person in need thereof.

The MS associated disease can be selected from the group consisting of Relapsing Remitting MS (RRMS), Secondary Progressive MS (SPMS), Primary Progressive MS (PPMS), Optic Neuritis (ON), Clinically Isolated Syndrome (CIS), and Acute Optic Neuritis (AON). In one particular embodiment is the disease is Secondary Progressive MS (SPMS) and/or Optic Neuritis (ON), Amyotrofic lateral sclerose (ALS).

### DETAILED DESCRIPTION OF THE INVENTION

An upregulation of molecules involved in lipid metabolism have been found in multiple sclerosis lesions. One variant of CPT1, CPT1a, is abundantly expressed in the brain and most organs where it catalyses the rate-limiting step in lipid metabolism. In MS lesions CPT1a expression has been found to be upregulated. Specifically CPT1a is an interesting molecule since this is a molecule which is a key molecule in lipid metabolism where upregulation of this molecule in MS correlates with a drop in lipid levels of MS patients due to an increased beta oxidation.

Blocking or downregulating the CPT1 and specifically CPT1a function will have therapeutic as well as prophylactic efficacy in multiple sclerosis. In addition, an efficacy of this approach can explain the inefficiency of an anti-inflammatory treatment of progressive MS as well as a lack of therapeutic efficacy of Multiple sclerosis in general.

Changes in the balance between sugar and lipid metabolism lead to a shift in the network connected to these changes resulting in a deterioration of the disease process. Processes where lipids play an important role are of course the functioning of the myelin sheet where the lipids are a crucial part. Loss of lipids in the myelin sheet result in a loss of function or reduced functioning of the myelin sheet; an increased signalling time, and increased energy expenditure.

In addition, it is hypothesized that the lipids have a protective function for the proteins of the myelin sheet. The myelin sheet proteins are post translationally modified where arginine's are deimidated and modified into citrullines. This modification makes the myelin sheet proteins very immunogenic. In addition will lipid metabolism under hypoxia result in prostaglandin production thereby attracting cells of the immune system to the exposed myelin sheet proteins. This process could explain the induction of the inflammatory response seen in MS patients.

The present inventors have surprisingly found that oral administrated of etomoxir, and in particular R(+)-etomoxir ethyl ester, can selectively downregulate CPT1 activity, and particularly CPT1a activity. The examples supports these findings.

The inventors have also surprisingly found that R(+)-etomoxir ethyl ester and specific administration patterns using R(+)-etomoxir ethyl ester allows for the use in the treatment, prevention and/or amelioration of a brain disease linked to lipid metabolism in a human individual, including MS and MS associated diseases. Thus, in its broadest aspect, the present invention relates to etomoxir with the chemical formula 2-[6-(4-chlorophenoxy)hexyl]-oxirane-2-carboxylic acid, for use in the treatment, prevention and/or amelioration of a brain disease linked to lipid metabolism in a human individual.

These brain diseases linked to lipid metabolism are also referred to herein as disorders caused by delipidation of neural tissue.

An aspect of the present invention relates to etomoxir with the chemical formula 2-[6-(4-chlorophenoxy)hexyl]-oxirane-2-carboxylic acid for use in the preparation of a medicament for the treatment, prevention and/or amelioration of a brain disease linked to lipid metabolism in a human individual.

Etomoxir of the present invention can exert its effect of the brain diseases in several ways. In one embodiment of the present invention is the treatment or amelioration done by downregulation of one or more cytokines selected from the group consisting of IL17a, TNFa, and IFNg.

IL17a is also found important in Rheumatoid Arthritis, Psoriasis, and other systemic Autoimmune diseases. Thus one embodiment of the present invention relates to etomoxir of the present invention for use in the treatment of rheumatoid arthiritis and/or psoriasis. Another embodiment of the present invention relates to the use of etomoxir of the present invention for use in the treatment of an autoimmune disease.

### 2-[6-(4-Chlorophenoxy)Hexyl]-Oxirane-2-Carboxylic Acid

2-[6-(4-chlorophenoxy)hexyl]-oxirane-2-carboxylic acid also known as etoxomir. Etomoxir is an irreversible inhibitor of carnitine palmitoyltransferase-1 (CPT-1) on the outer face of the outer mitochondrial membrane. This prevents the formation of acyl carnitines, a step that is necessary for the transport of fatty acyl chains from the cytosol into the intermembrane space of the mitochondria. This step is essential to the production of ATP from fatty acids in beta oxidation.

There are several versions of etomoxir. A preferred embodiment is the isomer R(+)-etomoxir with the chemical formula R(+)-2-[6-(4-chlorophenoxy)hexyl]-oxirane-2-carboxylic acid. In one embodiment of the present invention is the etomoxir racemic with the chemical formula Rac-2-[6-(4-chlorophenoxy)hexyl]-oxirane-2-carboxylic acid.

In an embodiment of the present invention is etomoxir the ethyl ester with the chemical formula 2-[6-(4-chlorophenoxy)hexyl]-oxirane-2-carboxylic acid ethyl ester, and in particular the isomer R(+)-2-[6-(4-chlorophenoxy)hexyl]-oxirane-2-carboxylic acid ethyl ester.

Etomoxir can also be the sodium salt hydrate with the chemical formula 2-[6-(4-chlorophenoxy)hexyl]-oxirane-2-carboxylic acid sodium salt hydrate, and the isomer R(+)-2-[6-(4-chlorophenoxy)hexyl]-oxirane-2-carboxylic acid sodium salt.

Importantly have the most published articles and experiments on etomoxir been focussed on etomoxir sodium salt. These includes for example Liu et al., BMC Cancer201212:154, Hernlund et al., Volume 123, Issue 2, 15 Jul. 2008 Pages 476-483, Gao et al., Biochemical Journal May 1, 2011, 435 (3) 723-732, and importantly Shriver et al., Scientific Reports 1, Article number: 79 (2011). It is worth noting that Shriver et al. uses administration of 15 mg/kg i.p. administration of etomoxir sodium salt. Thus, many experiments performed, including those made in preclinical studies on MS have also been using etomoxir sodium salt and/or racemic etomoxir where the (+). and (−)-isomers are mixed. Racemic etomoxir is often referred to simply as “etomoxir”.

The examples show for the first time the effects of certain concentrations of R(+)-etomoxir ethyl ester, i.e. R(+)-2-[6-(4-chlorophenoxy)hexyl]-oxirane-2-carboxylic acid ethyl ester. It is clear from example 1 that oral administrated R(+)-etomoxir ethyl ester can cross the blood-brain-barrier (BBB), from example 2 that R(+)-etomoxir ethyl ester has an effect in a MS mouse model, and from example 3 that R(+)-etomoxir ethyl ester has an improved effect on CPT1a. It therefore seems as if R(+)-etomoxir ethyl ester is advantageous in the treatment of disorders caused by delipidation of neural tissue.

This means that the concentration that the present inventors are using for the design of human experiments (clinical trials) could be estimated based on examples 1-3. The design of the clinical trials can be seen in examples 4 and 5. There are indications suggesting that 80 mg/day given once or twice (i.e. two times 40 mg/day) is the optimal dosage for treating MS or an MS associated disease, tested in example 4 on SPMS and example 5 on OP. Such dose is very surprising because the same dose previously has been used in a discontinued phase II clinical trial on congestive heart failure (due to adverse effects, Holubarsch et al., Clinical Science Aug. 1, 2007, 113 (4) 205-212;), and because other studies such as Shriver et al. has shown that a much higher dose should be used.

### Brain Disease Linked to Lipid Metabolism

An aspect of the present invention relates to etomoxir for use according to the present invention, including medical uses or methods of preventing, ameliorating and/or treating disorders caused by delipidation of neural tissue. An embodiment relates to the specific diseases or disorders selected from the group consisting of MS, MS associated disease, depression, Alzheimer's, and Parkinson's.

A further embodiment relates to the specific diseases or disorders selected from MS or an MS associated disease. In another embodiment is the specific disease or disorder an MS associated disease. The MS associated disease can be one or more selected from the group consisting of Relapsing Remitting MS (RRMS), Secondary Progressive MS (SPMS), Primary Progressive MS (PPMS), Optic Neuritis (ON), Clinically Isolated Syndrome (CIS), and Acute Optic Neuritis (AON). In one particular embodiment is the disease is Secondary Progressive MS (SPMS), Amyotrofic lateral sclerose (ALS), Neuromyelitis optica (NMO), depression, and/or Optic Neuritis (ON).

Another embodiment of the present invention relates to treating and/or preventing mood disorders, including Manic episode, Bipolar affective disorder, Depression, Depressive episode, Recurrent depressive disorder and Persistent mood disorders such as Cyclothymia and Dysthymia. In a further embodiment, the disorder is neurotic, stress-related and somatoform disorders, including Phobic anxiety disorders such as Panic disorder, Obsessive-compulsive disorder, Reaction to severe stress and adjustment disorders, Dissociative conversion disorders and Somatoform disorders.

In a further aspect of the invention, the etomoxir according to invention may be used for treating and/or preventing disorders which are behavioural syndromes associated with physiological disturbances and physical factors, including disorders selected from Nonorganic sleep disorders, Sexual dysfunction and Eating disorders such as Anorexia nervosa and Bulimia nervosa. In a further aspect of the invention the disorder is disorders of adult personality and behaviour, such as Paranoid personality disorder, Schizoid personality disorder, Dissocial personality disorder, Emotionally unstable personality disorder, Histrionic personality disorder, Anankastic personality disorder, Anxious personality disorder, Dependent personality disorder, Habit and impulse disorders such as Pathological gambling, Pathological fire-setting, Pathological stealing and Trichotillomania. In a further aspect of the invention, the disorder is mental retardation, including mild, moderate, severe and profound mental retardation. In another aspect of the invention, the etomoxir may be used for treating diseases of the nervous system, including the disorders multiple sclerosis and autoimmune neuropathies. Further disorders which can be treated according to the invention are, for example, Guillian-Barre, encephalomyelitis, Senile plaque, brain tumors i.e. glioblastoma multiforme, Huntingdon disease, Lou Gehrig's disease, pain, chronic pain, myastemia gravis, Sjogren's syndrome, Tourette syndrome, peripheral neuropathy, occipital neuralgia, motor neurone disease, meningitis, Chronic Lyme's disease, Encephalitis, Schilder's disease or diffuse myelinoclastic sclerosis, Chronic Inflammatory Demyelinating Polyneuropathy, Cerebral atrophy, Acute disseminated encephalomyelitis, Attention-deficit hyperactivity disorder, Cataplexy, Fibromyalgia, General anxiety disorder, Hypersexuality, Impulse-control disorders, Narcolepsy, Obsessive-compulsive disorder, Panic disorder, Posttraumatic stress disorder, Premenstrual dysphoric disorder, Social phobia, Chronic pain, Intermittent explosive disorder, Substance abuse and addiction (including alcoholism), cancer, dementia.

Depression can be associated with MS or not. Thus, in one embodiment of the present invention is the etomoxir of the present invention used in the treatment of depression as such.

In another embodiment of the present invention is the etomoxir of the present invention used in the treatment of Relapsing Remitting MS (RRMS). In a further embodiment of the present invention is the etomoxir of the present invention used in the treatment of Secondary Progressive MS (SPMS). In yet another embodiment of the present invention is the etomoxir of the present invention used in the treatment of Primary Progressive MS (PPMS). In another embodiment of the present invention is the etomoxir of the present invention used in the treatment of Optic Neuritis (ON). In another embodiment of the present invention is the etomoxir of the present invention used in the treatment of Clinically Isolated Syndrome (CIS). In a further embodiment of the present invention is the etomoxir of the present invention used in the treatment of Acute Optic Neuritis (AON). In another embodiment of the present invention is the etomoxir of the present invention used in the treatment of Amyotrofic lateral sclerose (ALS). In another embodiment of the present invention is the etomoxir of the present invention used in the treatment of Neuromyelitis optica (NMO). In another embodiment of the present invention is the etomoxir of the present invention used in the treatment of Parkinson's.

The patient to be treated with the methods of the present invention is preferably human individual.

### Administration Form

In a further aspect of the invention there is provided medical uses or methods of preventing, ameliorating and/or treating disorders caused by delipidation of neural tissue, by administering etomoxir to a patient in need thereof in a pharmacologically effective amount.

As used herein, a “pharmaceutically effective amount” of etomoxir is an amount effective to achieve the desired physiological result, either in cells treated in vitro or in a subject treated in vivo. Specifically, a pharmaceutically effective amount is an amount sufficient to inhibit, for some period of time, one or more clinically defined pathological effects associated with disorders caused by delipidation of neural tissue.

The pharmaceutically effective amount may vary depending on a variety of factors and conditions related to the subject to be treated and the severity of the disease. For example, if the inhibitor is to be administered in vivo, factors such as age, weight, sex, and general health of the patient as well as dose response curves and toxicity data obtained in pre-clinical animal tests would be among the factors to be considered. Preferably, the inhibitor is present in a pharmaceutical composition and often in a concentration of 0.01 to 50% per weight of the pharmaceutical composition, more preferably 1 to 30%.

The pharmaceutical composition according to the invention can be administered in a conventional manner, e.g. by means of oral dosage forms, such as, for example, tablets, capsules, powder by means of the mucous membranes, for example the nose or the oral cavity, or gels which contain the pharmaceutical compositions according to the invention. Thus, an embodiment of the present invention relates to etomoxir for use according to the present invention, wherein etomoxir is formulated for oral administration. Another embodiment of the present invention relates to etomoxir for use according to the present invention, wherein etomoxir is formulated for oral administration as a tablet, powder or capsule. The pharmaceutical composition can also be comprised in a sticker, such as a patch.

In the context of the present invention, the CPT-I inhibitor may be administered as such, e.g. in substantially pure form, or preferably in combination with at least one excipient and/or auxiliary, e.g. with one or more suitable adjuvant(s) and/or one or more pharmaceutically active and/or acceptable carrier(s), diluent(s), filler(s), binder(s), disintegrant(s), lubricant(s), glident(s), coloring agent(s), flavoring agent(s), opaquing agent(s) and plasticizer(s).

In a further embodiment said prevention, amelioration, and/or treatment of disorder according to the invention comprises the administration of etomoxir in combination with a further therapy. This may result in an additive or even synergistic effect. Without being bound to any theory, the reason for the additive or synergistic effect might be that each therapeutic mean has its own mechanism, and the combination of different mechanism results in an additive or synergistic effect. Example of further therapy is selected from the group comprising inhibiting hormones or HMGCoA reductases, such as, for example statins like prolactin or somatostatin and chalones (mitotic inhibitors); especially mevastatin, lovastatin, simvastatin, pravastatin, fluvastatin and cerivastatin; fibrates, such as, for example, fenofibrate; clofibrate; clofibric acid derivatives, such as, for example, etofibrate, etofyllinclofibrate; clofibratanaloga, such as, for example, bezafibrate or gemfibrozil; steroids, especially cortisone, vitamin D or derivatives thereof, vitamin A or derivatives thereof, Vitamin B or derivatives thereof, especially vitamin B12, dithranol, urea, salicylic acid, Mahonia aquifolium, fumaric acid, fumaric acid esters, blockers of arachidonic acid, e.g. ometa-3 fatty acids, antibiotics, antimycotics, immunomodulators, e.g. methotrexate, cyclosporine, Fk506, E-selectin blockers, P-selectin blockers, ICAM blockers, LFA-I blockers, LFA-2 blockers, LFA-3 blockers, VCAM blockers, and/or TNF blockers, with cytokine inhibitors and T-cell activation inhibitors. The above blockers are e.g.

antibodies or competitive inhibitors of E-selectin, P-selectin, ICAM, LFA-I, LFA-2, LFA-3, VCAM or TNF, neurological modifiers i.e., acetylcholine receptor blockers, memantine or derivates thereof, galantamine or derivates thereof, Donezepil or derivates thereof, rivastigmine or derivates thereof, .beta nicotinamide adenine dinucleotide or derivates thereof, 5-Hydroxytryptamine (5-HT) Reuptake Inhibitor, Adenosine Al Receptor (ADORAI) Antagonist, Dopamine Reuptake Inhibitor, Estrogen Receptor 2 (ESR2) Agonist, Phosphodiesterase-4 (PDE-4) Inhibitor, Corticotropin-Releasing Factor Receptor 1 (CRFRI) Antagonist, Corticotropin-Releasing Factor Receptor 1 (CRFRI) Antagonist, Monoamine Oxidase B (MAO-B) Inhibitor, Norepinephrine Reuptake Inhibitor, 5-Hydroxytryptamine (5-HT) Reuptake Inhibitor, cannabinoid receptor inhibitor, Lingo-inhibitors.

It is recommended that patients with SPMS with evidence of relapses to be treated with Interferon Beta; however, in patients with SPMS but no evidence of active inflammation, there is no strong evidence of efficacy of any of the available therapies. Thus, in one embodiment of the present invention are the patients treated with Interferon Beta and etomoxir. One embodiment relates to anti-lingo, such as lingo-inhibitors combined with etomoxir for use in the treatment of the diseases mentioned herein.

### Dosage Regimen

The etomoxir of the present invention can be used for the different applications described herein at different concentrations. It is important that these concentrations are selected based to maximize the effect and minimize side effects.

Thus, an embodiment of the present invention relates to etomoxir for use according to the present invention, wherein etomoxir is administered according to an administration pattern comprising: administration of 40-120 mg/day to a person in need thereof. An embodiment of the present invention relates to etomoxir for use according to the present invention, wherein etomoxir is administered according to an administration pattern comprising: administration of 40 mg/day to a person in need thereof. An embodiment of the present invention relates to etomoxir for use according to the present invention, wherein etomoxir is administered according to an administration pattern comprising: administration of 50 mg/day to a person in need thereof. An embodiment of the present invention relates to etomoxir for use according to the present invention, wherein etomoxir is administered according to an administration pattern comprising: administration of 70 mg/day to a person in need thereof. An embodiment of the present invention relates to etomoxir for use according to the present invention, wherein etomoxir is administered according to an administration pattern comprising: administration of 80 mg/day to a person in need thereof. An embodiment of the present invention relates to etomoxir for use according to the present invention, wherein etomoxir is administered according to an administration pattern comprising: administration of 80 mg/day to a person in need thereof, wherein the 80 mg/day etomoxir is administered at two times 40 mg/day to a person in need thereof. An embodiment of the present invention relates to etomoxir for use according to the present invention, wherein etomoxir is administered according to an administration pattern comprising: administration of 90 mg/day to a person in need thereof. An embodiment of the present invention relates to etomoxir for use according to the present invention, wherein etomoxir is administered according to an administration pattern comprising: administration of 100 mg/day to a person in need thereof. An embodiment of the present invention relates to etomoxir for use according to the present invention, wherein etomoxir is administered according to an administration pattern comprising: administration of 120 mg/day to a person in need thereof. An embodiment of the present invention relates to etomoxir for use according to the present invention, wherein etomoxir is administered according to an administration pattern comprising: administration of 80 mg/day to a person in need thereof. A preferred embodiment of the present invention relates to R(+)-etomoxir ethyl ester with the chemical formula R(+)-2-[6-(4-chlorophenoxy)hexyl]-oxirane-2-carboxylic acid ethyl ester, for use in the treatment or amelioration of Multiple

Sclerosis (MS) or an MS associated disease, wherein the R(+)-etomoxir ethyl ester is administered according to an administration pattern comprising: administration of 80 mg/day to a person in need thereof. Another preferred embodiment of the present invention relates to etomoxir for use according to the present invention, wherein the 80 mg/day R(+)-etomoxir ethyl ester is administered at two times 40 mg/day to a person in need thereof.

### Method of Treatment

An aspect of the present invention relates to a method for treating, preventing and/or ameliorating a brain disease linked to lipid metabolism, comprising administration of etomoxir with the chemical formula 2-[6-(4-chlorophenoxy)hexyl]-oxirane-2-carboxylic acid to a human individual in need thereof.

Another aspect of the present relates to etomoxir with the chemical formula 2-[6-(4-chlorophenoxy)hexyl]-oxirane-2-carboxylic acid for use in the preparation of a medicament.

### Test

An embodiment of the present invention relates to etomoxir for use according to the present invention, wherein the treatment or amelioration is measured as a statistically significant change in Normalized Brain Volume (NBV) over a period of 6 months compared to placebo. In a preferred embodiment is NBV correlated with SPMS.

An embodiment of the present invention relates to etomoxir for use according to the present invention, wherein the treatment or amelioration is measured as a statistically significant change in a parameter selected from the group consisting of change in RNFL/GCL-IPL thickness at month 18 compared to baseline, MSFC z-score; EDSS change, Number of new T2-hyperintense lesions at month 6 evolving into persistent T1 hypointense lesions (black holes) at month 18, Leg MEP/TMS (latency, amplitude), and Neurofilaments (CSF/serum) and/or other suitable biomarkers. In a preferred embodiment is one or more of these correlated with SPMS.

An embodiment of the present invention relates to etomoxir for use according to the present invention, wherein the treatment or amelioration is measured as a statistically significant change in affected minus baseline affected eye GCL-IPL (ganglion cell layer plus inner plexiform layer) thickness. In a preferred embodiment are these correlated with OP.

An embodiment of the present invention relates to etomoxir for use according to the present invention, wherein the treatment or amelioration is measured as a statistically significant change in a parameter selected from the group consisting of change in RNFL/GCL-IPL thickness at month 12 compared to baseline, Low-contrast letter visual acuity, QoL self-rating scale, combined unique active (CUA) lesions on follow-up scan (month 6)(no. of new or newly enlarging T2 lesions, Gd+ lesions). No. of T2-hyperintense lesions evolving into persistent T1 hypointense lesions (black holes) at month 12, or VEP (P100 latency and P100 amplitude). In a preferred embodiment is one or more of these correlated with OP.

An embodiment of the present invention relates to etomoxir for use according to the present invention, wherein the treatment or amelioration is measured as a statistically significant change in one or more parameters selected from the group consisting test on Function, test on inflammatory response, est on vision, test on disability, test on autoantibodies, test on auto cells, test on lipid levels in the body, test on lipid levels in the brain, and test on Gd+ lesions

### General

It should be understood that any feature and/or aspect discussed above in connections with the compounds according to the invention apply by analogy to the methods described herein.

The following figures and examples are provided below to illustrate the present invention. They are intended to be illustrative and are not to be construed as limiting in any way.

### BRIEF DESCRIPTION OF THE FIGURES

FIG. 1 shows that R(+)-etomoxir ethyl ester induces an increased sucrose consumption. Blocking CPT-1 in hypothalamus (intracranial injection) increases food intake. The testing period is 20 days.

FIG. 2 shows that R(+)-etomoxir ethyl ester induces restores in 40% of the animal's long term memory.

FIG. 3 shows the different IC50 values for MCPT1 (CPT1b) vs LCPT1 (CPT1a).

FIG. 4 shows the study synopsis for example 3. MSFC=Multiple Sclerosis Functional Composite; EDSS=Expanded Disability Status Scale; OCT=Optical coherence tomography.

FIG. 5 shows the brain atrophy measures in different MS subtypes.

FIG. 6 shows the study synopsis for example 4. VEP=Visual evoked potential; VA=Low contrast letter visual acuity; QoL=Quality of life (visual); OCT=Optical coherence tomography.

Comparison over time of the efficacy of R(+)-etomoxir ethyl ester and Escitalopram in chronic mild stress-induced depression.

FIG. 8

Percentage of responding animals in four weeks of treatment with R(+)-etomoxir ethyl ester. Etomoxir shows high efficacy compared to Escitalopram.

FIG. 9

Human peripheral blood lymphocytes stimulated with staphylococcal enterotoxin B (SEB) for 48 h and gated for CD3+ cells. SEB stimulation activates the immune system. Treatment with Etomoxir revealed downregulation of IFNgamma, IL-17alpha and TNF-alpha. The % of cells responding to SEB is set at 100% and the % of cells present when unstimulated is set at 0%.

FIG. 10

Two weeks of treatment 1 mg/kg/day sc. R(+)-etomoxir ethyl ester injection shows statistical significant clinical efficacy in the mouse EAE model. At the end of study 52% of the Etomoxir treated animals are symptom free (a, p=0.009),

FIG. 11

Test of the therapeutic efficacy for the treatment of EAE in vivo, we also tested the treatment with R(+)-etomoxir ethyl ester in rat models. The treatment of the animals with Etomoxir or placebo started at day 7 after induction at the end of the study 25% of the animals were symptom free.

FIG. 12

R(+)-etomoxir ethyl ester treatment demonstrated significantly better therapeutic effects compared to IFNb when it was started at day 1 or day 5.

FIG. 13

R(+)-etomoxir ethyl ester restores the animal's short term memory, and confirms that R(+)-etomoxir ethyl ester is effective in the treatment of amelioration on MS or MS associated diseases.

### EXAMPLES

### Example 1

### Etomoxir Goes to the Brain After Drinking the Drug

### Study Design

First rats are given R(+)-etomoxir ethyl ester formulated water (4 mg/day) every day for 20 days. Then once a week the sucrose consumption was measured. This study is designed to test is R(+)-etomoxir ethyl ester reaches the brain and therefore is it can cross the blood-brain-barrier (BBB).

### Results

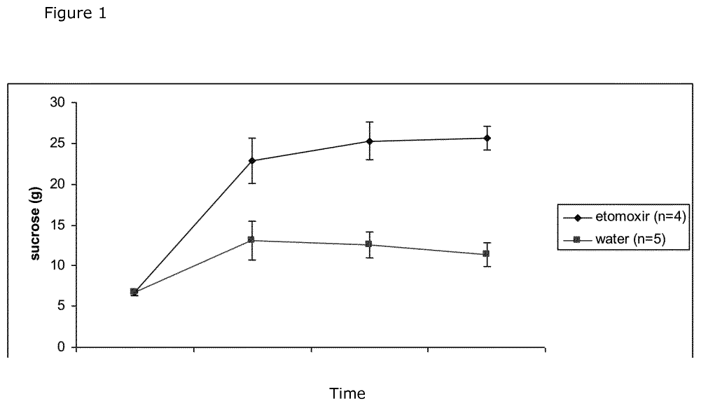

FIG. 1 shows that R(+)-etomoxir ethyl ester induces an increased sucrose consumption. Blocking CPT-1 in hypothalamus (applied in the drinking water) increases food intake.

Thus, R(+)-etomoxir ethyl ester can cross the blood-brain-barrier (BBB) and reach the brain.

### Example 2

### Effect of Etomoxir on Long Term Memory

### Study Design

Rats are put in a cage where in the dark part (where they like to be) the bedding gives a shock. They are put in the light part during training. The next day the rats are put in the light part again, when long term memory is there, they will not like to go in the dark part (shock compartment).

MK801, blocks the generation of memory.

The test of R(+)-etomoxir ethyl ester with or without MK801 in this setup shows whether R(+)-etomoxir ethyl ester can restore long term memory, and serve as a model for the effect on MS or MS associated diseases.

Etomoxir was given by sub cutaneous injection 1 hour before the test. This shows that Etomoxir can cross the blood brain barrier within 1 hour and exert its function.

### Results

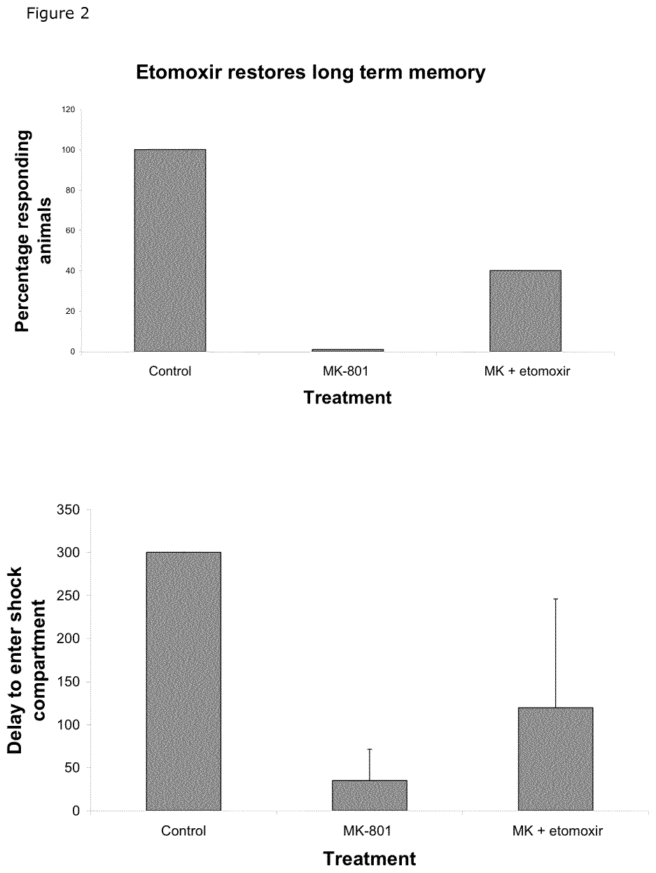

FIG. 2 shows that R(+)-etomoxir ethyl ester induces restores in 40% of the animal's long term memory, and confirms that R(+)-etomoxir ethyl ester is effective in the treatment of amelioration on MS or MS associated diseases.

### Example 3

### IC50 Values for R(+)-Etomoxir Ethyl Ester on MCPT1(CPT1b) vs LCPT1(CPT1a)

### Study Design

CPT1a and CPT1b protein was expressed in yeast cells and purified from the yeast cells.

After purification C¹⁴ labelled R(+) Etomoxir was added to the individual proteins and half life of binding of the radioactive Etomoxir to CPT1a/CPT1b was measured.

### Results

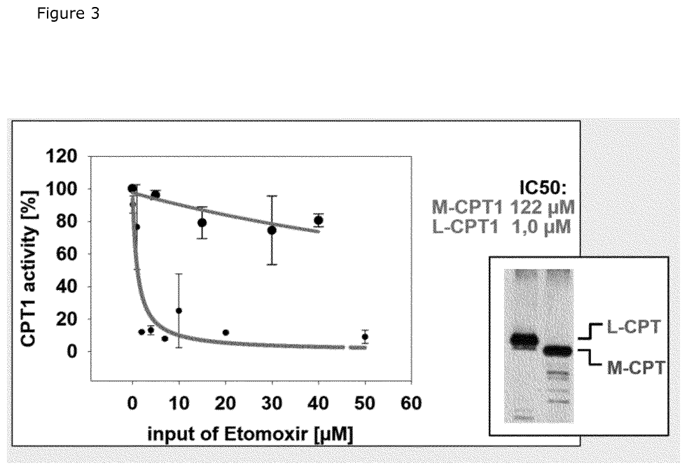

The IC50 values of R(+)-etomoxir for MCPT1 (CPT1b) vs LCPT1 (CPT1a) are shown in FIG. 3.

R(+)-etomoxir clearly has a much better inhibitory effect on CPT1a than CPT1b.

### Example 4

### Etomoxir and SPMS

### Background

Multiple sclerosis is an inflammatory, demyelinating and degenerative disease of the central nervous system (CNS). Brain atrophy accrues throughout the disease but commences already at the earliest disease stages, i.e. clinically isolated syndrome (CIS). Performing a proof-of-concept trial to show efficacy of a neuro- or myelin-protective treatment based on a clinical end-point during chronic stages of MS is not feasible from several reasons, the main ones being the size and length of the trial. Therefore one has to resort to surrogate end-points that are based on imaging or other outcomes, which are indicative of efficacy and serve as the basis for further clinical development. Regarding potential surrogate outcomes correlations of structural imaging and clinical signs are overall weak. However, among the possible measures those that capture atrophy of both global (brain parenchymal fraction, BPF) and focal (black holes, BH) tissue destruction appear more informative than markers of inflammation. Available disease-modifying treatments target the inflammatory component of MS and have little if any effect on degenerative aspects and disability evolution that characterize progressive forms of the disease.

Based on the above considerations structural MRI measures of brain tissue volume have become the preferred surrogate measure of MS disease progression. Estimates of global brain atrophy rates vary in studies that directly compared different MS disease subtypes. Based on previous findings however, atrophy without prominent inflammation is supposed to be more pronounced in progressive—as compared to relapsing remitting forms of the disease (relapsing remitting MS, RRMS). Therefore, progressive MS subtypes (secondary progressive MS, SPMS; primary progressive MS, PPMS) have been proposed as models for clinical trials testing compounds claiming neuroprotective potential.

### Study Design

In order to allow for reliable assessment of longitudinal brain tissue loss the follow-up has to cover at least a period of 12-18 months. In addition, to avoid a major confound by anti-inflammatory effects of a compound, which leads to so-called “pseudo-atrophy” and masks potential neuroprotective effects, the reference scan should be performed at least three to six months from baseline and start of the study drug. Co-registration of two consecutive MRIs at reference visits may increase reliability of MRI atrophy measures, however, this aspect has not yet been formally addressed.

Based on these findings the following phase IIa proof-of-concept study protocol for tissue protection by Etomoxir in patients with SPMS is performed:

### Study Design.

Randomized, placebo-controlled, double-blind, parallel-groups study.

### Study Duration.

Study duration is 18 months overall. Reference MRIs are performed 6 months post baseline and at study end at month 18.

### Study Cohort.

Secondary progressive MS patients (aged 18-60) with an EDSS between 4.0 and 6.5. Patients had to have disability progression not related to relapse activity in the year prior to study inclusion. Disability progression should be documented by an EDSS increase of 1.0 in case the EDSS had been <5.0, or 0.5 in case the EDSS had been ≥5.0, respectively.

### Primary Endpoint.

Rate of change of normalized brain volume (NBV) or brain parenchymal fraction (BPF) over 12 months (baseline+6 months compared to month 18).

### Secondary Endpoints.

a. OCT. Change in RNFL/GCL-IPL thickness at month 18 compared to baseline.
b. Clinical. MSFC z-score; EDSS change.
c. Radiological. Number of new T2-hyperintense lesions at month 6 evolving into persistent T1 hypointense lesions (black holes) at month 18.
d. Electrophysiology. Leg MEP/TMS (latency, amplitude).
e. Exploratory: Neurofilaments (CSF/serum) and/or other suitable biomarkers

Study drug. Patients are randomized 1:1 into the following two study arms: 1. study drug (R(+)etomoxir ethyl ester). 2. placebo. Administration of study drug (dose, frequency).

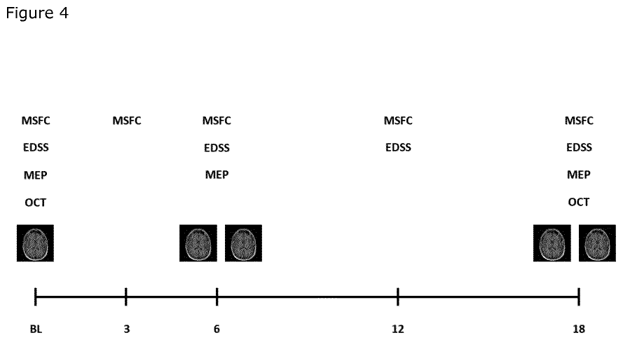

The study synopsis can be seen in FIG. 4.

### Sample Size Estimation.

The sample size to give a statistical power of 80% at a significance level of 5% for a treatment effect of 50% on the primary endpoint (NBV/BPF change rate at month 18 compared to month 3/6) with a provided drop-out rate of 20% would be 78. (Based on an effect size estimate of 30% whilst otherwise unchanged conditions the sample size would increase to 204 patients).

### Results

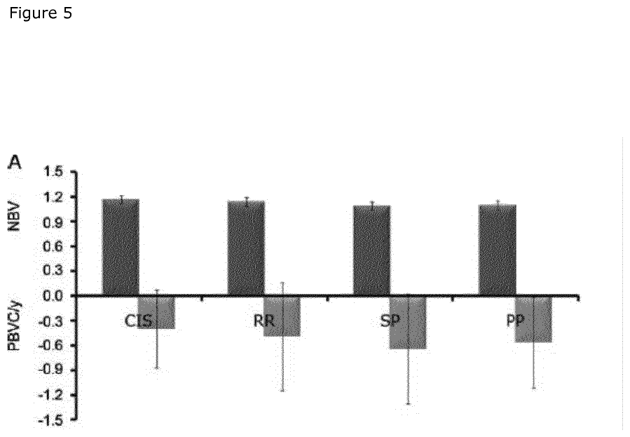

Atrophy in MS patients as assessed by the yearly parenchymal brain volume change (PBVC/y) is up to 5-10-fold higher than the atrophy rate that is expected by normal ageing (0.5%-1.0% compared to 0.1%), and the most prominent changes are observed in SPMS patients (see FIG. 5).

There are indications suggesting that the effect of R(+)-etomoxir ethyl ester is to reverse or ameliorate the atrophy or atrophy rate.

There are indications suggesting that 80 mg/mL/day in one or two tablets of R(+)-etomoxir ethyl ester is preferred.

### Example 5

### Etomoxir and Optic Neuritis (OP)

### Background

Optic neuritis (ON) is a clinical hallmark of multiple sclerosis (MS) both at primary manifestation—the so-called clinically isolated syndrome (CIS)—and during the relapsing remitting course of the disease (relapsing remitting multiple sclerosis, RRMS) after confirmation of diagnosis.

42% of patients suffering from ON during their disease have residual deficits large enough to be EDSS relevant. Optical coherence tomography (OCT) is a novel and innovative tool increasingly recognized in MS research for its potency to capture degenerative changes in the retina of MS patients following episodes of acute ON but also irrespective thereof, reflecting degeneration remote of sites of acute autoimmune inflammation. In addition, there is remarkable association with measures of brain atrophy in MS as assessed by MRI.

During the acute phase of ON swelling of the optic disc caused by inflammatory oedema can be observed in some but not all patients with ON which may mask early atrophy and confound baseline OCT measurements. However, 1-2 months after ON onset significant inter-eye differences (affected vs. unaffected) become overt. At least half of the final thinning related to the acute episode becomes evident by month 3. The median loss of retinal nerve fibre layer (RNFL) thickness associated with a single episode of ON is 15-20% of the baseline value (15-20 μm).

While almost the whole loss in RNFL is predicted to appear until month 6 after ON onset, subtle changes may still occur until month 12 but rarely—if at all—later than that.

Recently, segmentation algorithms have become available for the segmentation of retinal layers below the level of retinal ganglion cell axons. We together with collaborating centres have pioneered pilot studies in CIS patients proving early atrophy of retinal layers beyond the RNFL. Preliminary, including our own data suggest that, in ON-CIS, macula-centred segmentation of the retina may provide a secondary or even co-primary endpoint in an interventional trial in the disease model discussed here. Ganglion cell layer (GCL) atrophy has previously been shown to correlate strongly with visual outcome after acute ON. In addition, GCL-segmentation is not confounded by inflammatory oedema in the acute phase of ON.

Based on these findings the following phase IIa proof-of-concept study protocol for tissue protection by Etomoxir in patients with an episode of acute ON is tested.

### Study Design

Study Design. Randomized, placebo-controlled, double-blind, parallel-groups study.

Study Duration. Core study is a 6 month study with an additional and final follow-up at month 12 after onset of acute ON.

Study cohort. Patients (aged 18-55) with a first episode of acute (≤7 days of symptom onset) unilateral ON either suggestive of Multiple Sclerosis (at least 1 white matter lesion in typical location of ≥2 mm and/or positive CSF*) or in patients with a confirmed diagnosis of RRMS not on immunomodulatory and/or experimental treatment and without a history of previous optic neuritis on either eye. Primary endpoint. Standard deviation (SD) of mean difference of follow-up (month 6) affected- minus baseline fellow eye RNFL.

Co-primary endpoint. Mean follow-up affected minus baseline affected eye GCL-IPL (ganglion cell layer plus inner plexiform layer) thickness.

### Secondary Endpoints.

a. OCT. Change in RNFL/GCL-IPL thickness at month 12 compared to baseline.
b. clinical. Low-contrast letter visual acuity. Visual QoL self-rating scale.
c. radiological. Combined unique active (CUA) lesions on follow-up scan (month 6)(no. of new or newly enlarging T2 lesions, Gd+ lesions). No. of T2-hyperintense lesions evolving into persistent T1 hypointense lesions (black holes) at month 12.
d. Electrophysiology. VEP (P100 latency and P100 amplitude).

Study drug. Patients are randomized 1:1 into the following two study arms: 1. steroids+study drug (etomoxir). 2. steroids+placebo. Administration of study drug (dose, frequency) tbd.

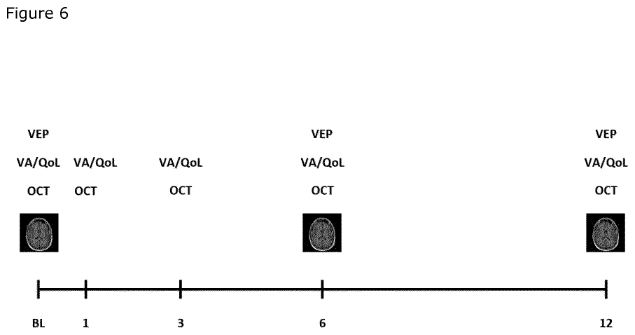

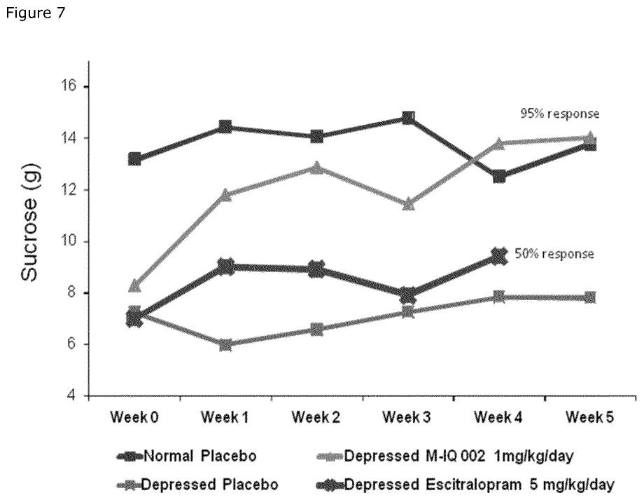

Study synopsis can be seen in FIG. 6.

### Sample Size Estimation.

For the primary endpoint (RNFL) based on an estimated effect size of 50% on the decrease of RNFL loss at month 6 with a power of 80%, a type 1 error of 0.05 and a provided drop-out rate of 20% approximately 48 patients is randomized (effect size of 30% while otherwise unchanged conditions would need approx. 120 patients to be randomized).

### Results

There are indications suggesting that R(+)-etomoxir ethyl ester has an effect in line with the above protocol.

There are indications suggesting that 80 mg/mL/day in one or two tablets of R(+)-etomoxir ethyl ester is preferred.

### Example 6

### Effect of Etomoxir on Depression

### Results

Expression of carnitine palmitoyl transferase la mRNA in depressed patients The expression of CPT1a mRNA in pathological brain samples of patients that committed suicide with a history of depression and compared to healthy donors was analyzed by affimetrix analysis. Results from this analysis showed a significant upregulation of CPT1a expression in cerebellum (p=0.0021).

Treatment of stress-induced depression by blocking carnitine palmitoyl transferase 1 Rats were initially exposed to four weeks of CMS and subsequently exposed to stressors for another five weeks combined with drug or vehicle treatment (FIG. 7). The intake of sucrose solution was used in order to determine the depression status among rats. There was no significant difference between rats receiving vehicle treatment compared to groups receiving Escitalopram. In the Etomoxir treated group of rats, while exposed to stress for five weeks, and compared to a vehicle group exposed to stress and an unchallenged control group, the intake of sucrose solution was significantly higher in rats treated with Etomoxir compared to the vehicle group in all weeks. Moreover, statistical significant difference was found between Escitalopram and Etomoxir treatment in week two, four and five.

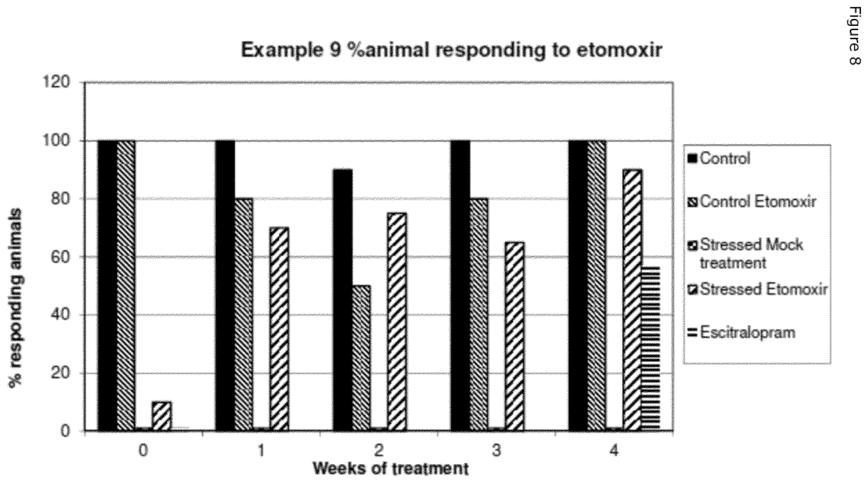

Treatment with Etomoxir during CMS exposure increased the level of sucrose intake to the same level as for unchallenged rats after five weeks of treatment. No significant differences were observed between unchallenged controls receiving vehicle or Etomoxir and stress exposed rats receiving Etomoxir. The percentage of animals responding over time receiving Etomoxir, Escitalopram or vehicle was compared (FIG. 8). The criterion for responders was set at 20% increase in intake of sucrose at the respective weeks compared to baseline prior to onset of treatment. The percentage of responding rats to Etomoxir treatment was higher than Escitalopram treatment.

Escitalopram treatment for 5 weeks showed 57% healthy rats with continued exposure to stress (FIG. 8) which is not significant compared to vehicle. Etomoxir was efficacious in 90% of the rats compared to baseline, which was statistically highly significant (p=0.0007).

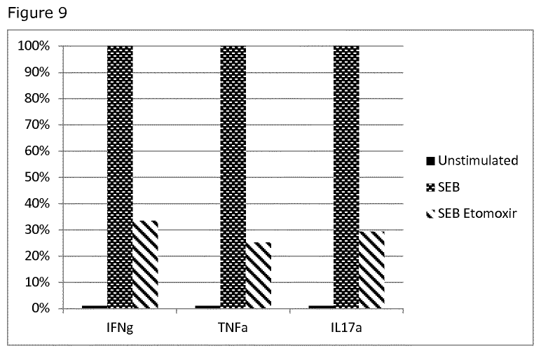

The effect of carnitine palmitoyl transferase 1 blockage on the immune system Human peripheral blood lymphocytes were stimulated with the T-cell activating agent, streptococcal enterotoxin B (SEB) for 48 hours in order to activate the immune system (FIG. 9). An unstimulated group and a SEB stimulated group were treated with Etomoxir. The human peripheral blood lymphocytes were gated for CD3+ cells. Unstimulated lymphocytes receiving Etomoxir and no SEB treatment showed low production of all cytokines. SEB stimulated lymphocytes receiving Etomoxir treatment revealed 67%, 71% and 75% downregulation of interferon-gamma (IFN-gamma), interleukin-17alpha (IL-17alpha) and tumor necrosis factor-alpha (TNF-alpha). The number of SEB stimulated cells was set at 100% the number of unstimulated cells at 0%. The effect of Etomoxir was calculated relative to these 2 values.

### Methods

### Affimetrix Analysis

Tissue from patients or healthy donors has been isolated according to Gene Logic protocols. Afterwards the mRNA was analyzed for expression of CPT1a in affimetrix analysis Genbank ID: NM_001876, and expression was analyzed with GeneExpress® and e-Northern™ proprietary informatics programs of Gene Logic.

### Animals

Male Wistar rats were used for the CMS model. The weight of the rats was approximately 200 g when the experiment was initiated. The rats were housed singly with 12 h light/dark cycle and food and water was available ad libitum except when these parameters were applied as stress inducers. The following paragraphs concerning the CMS model was performed according to the protocol by Wiborg et al.

### Sucrose Consumption Test

In order to quantify the hedonic state of the animals a sucrose consumption test was performed. The animals were trained in five weeks in order to consume a palatable sucrose solution.

In the period of the five weeks training, the animals were tested twice a week during the first three weeks and only once a week the last two weeks. The animals were deprived for food and water in 14 h before the sucrose consumption test. The test involved 1 h access to a 1.5% sucrose solution. When the stress period was initiated the sucrose consumption test was performed once a week.

### Chronic Mild Stress Protocol

The animals were divided into two groups; one group was exposed to stress and one control group was unchallenged. The two groups were matched in such a manner that both mean and standard deviation in sucrose consumption were similar. The animals were then placed in separate rooms. One group was exposed to an initial four week period of chronic mild stressors, while the other group was left undisturbed. Food and water was freely available for the unchallenged group, except 14 h before the sucrose consumption test where the animals were food and water deprived. The stress paradigm persisting in four weeks involved one period of intermittent illumination, stroboscopic light, grouping of the animals, and food and water deprivation. Moreover, there were two periods with no stress and soiled cage and three periods of tilting the cage 45°.

### Drug Administration

After four weeks of CMS exposure, the animals were treated with either drug or vehicle for five weeks while still exposed to stressors. Etomoxir (Meta-IQ ApS, Denmark), a specific CPT1 inhibitor, was heated to 37° C. and dissolved in sunflower oil. Etomoxir was administrated intraperitoneally every day in a dosage of 4 mg/kg. Escitalopram (Lundbeck, Denmark) was dissolved in saline and was administrated intraperitoneally daily in a dosage of 5 mg/kg.

### Intracellular Staining for Flow Cytometry

Blood lymphocytes were isolated from humans using a buffy coat. Sodium heparin full blood was centrifuged at 2500 rpm for 15 min and the white blood cell layer was harvested afterwards the cells were centrifuged over a Ficoll density gradient at 2000 g for 10 min. The white blood cells were harvested, centrifuged at 2000 rpm for 5 min and the supernatant was discarded. The cells were plated in 6 well plates 2 mill. cells pr. well and cultured for 48 h. in RPMI medium containing 10% fetal calf serum and 1% penicillin/streptomycin in the presence or absence of staphylococcal enterotoxin B (30 ng/ml). Etomoxir treated cells were additionally cultured with Na-Etomoxir (100 μM).

After 48 h, the cells were washed in phosphate buffer saline (PBS)/bovine serum albumin (BSA). The cells were stained for APC mouse anti-human CD3, CD4 Per CP-Cy in PBS/BSA and incubated on ice for 1 h. The staining procedure was carried out according to Intracellular Staining Kit (Invitrogen). The antibodies FITC mouse anti-human IFN-gamma, PE mouse anti-human IL-4, PE mouse anti-human IL-17alpha and FITC mouse anti-human TNF-diluted in permeabilization buffer and incubated on ice for 30 min. The cells were washed twice and re-suspended in PBS for further analysis using flow cytometry.

### Example 7

### Effacy of Etomoxir in EAE Rat and Mouse Model for MS

### Introduction

The present example shows testing of the efficacy of a CPT1 blocker in treating advanced experimental allergic encephalitis (EAE) a recognized animal model for MS, as well as analyze CPT1a expression in animal EAE as well as human MS lesion. As a last part we screened the genome database for mutations in CPT1a and multiple sclerosis prevalence.

### Materials and Methods

### Animals

Six week old C57B16 mice were bred and kept at conventional animal facilities at the University of Copenhagen. Female Lewis rats, 2 months old were used for this study.

All animals were weighed and scored clinically on a daily basis. During progression of EAE, leading to motor disabilities, animals had water in a Petri dish and soaked chow for easy intake to ensure sufficient intake of liquid and nutrients. Healthy animals were used as comparison and control of the EAE exposed animals.

### EAE Induction

In the rat, EAE was induced by intradermal (i.d.) injection of 0.2 mL of an emulsion consisting of 100 μg Myelin Basic Protein Histochem Cell Biol 123 (MBP) from guinea pig (Sigma, USA, M2295) suspended in 0.1 mL of saline (0.9%) and 0.1 mL complete Freund's adjuvant (CFA; Difco, USA, 0638), with the addition of 0.2 μg of Mycobacterium Tuberculosis H37 Ra (Difco, USA, 3114). This emulsion was given intradermally at the base of the tail of each rat in the EAE groups under Isoflurane anesthesia (Isoflurane Baxter, Baxter, USA, KDG9623).

In the mouse, C57BI6 mice were immunized subcutaneously in the flanks with 200 μg of MOG 35-55 peptide in 0.1 mL PBS and 0.1 mL CFA containing 0.4 mg Mycobacterium tuberculosis (H37Ra; Difco Laboratories, Detroit, Mich., USA) and intraperitoneally injected with 200 ng Pertussis toxin (List Biological Laboratories Inc., Campbell, Calif., USA) on the day of immunization and 2 days later.

### Treatment:

At indicated time points, animals were treated either with saline or olive oil (both placebo groups), or etomoxir ethyl ester 1 mg/kg/day diluted in olive oil at 370 C or IFNγ 200,000 IU every other day.

### Score of Clinical Symptoms:

The animals were monitored daily, weighed and clinically scored according to the following scale (Imrich and Harzer 2001): 0, no clinical signs of EAE; 1, loss of tail tonus; 2, mild paresis in one or both hindlimbs; 3, moderate paresis in one or both hindlimbs; 4, severe paresis or paralysis in one or both hindlimbs; 5, paralysis in one or both hindlimbs and visible paresis in one or both forelimbs, incontinence; 6, moribund. (Wiskin et al. 2010).

### Results

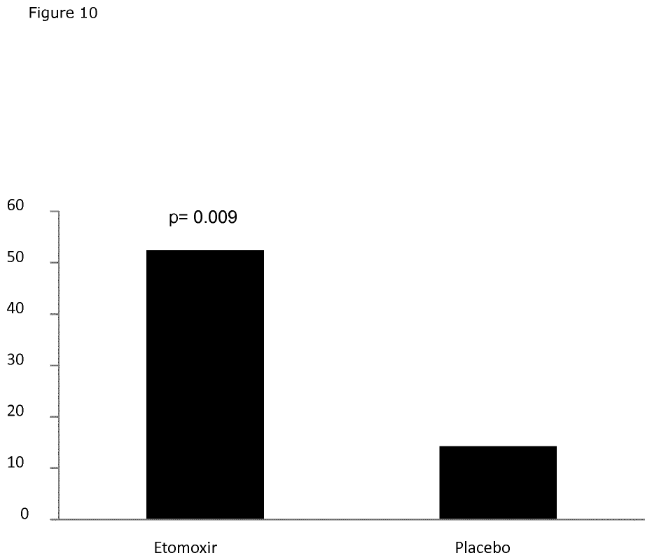

CPT1 Blockers Have a Therapeutic Effect in Rodent Models with EAE. CPT1a is upregulated in lesion with MS/EAE. For examining the therapeutic efficacy of the drug Etomoxir in a therapeutic setting. This means that we first induced the disease by immunization of C57B16 mice with the MOG 35-55 peptide emulsified in Complete freund's adjuvant (CFA) containing 400 ng pertussis toxin. Ten days after injection of the MOG peptide, the animal started showing sign of laming of the tail and/or hind legs. At this time point, we started the treatment of the animals through injection s.c. either with 1 mg/kg Etomoxir ester in olive oil or placebo (olive oil alone). The animals were daily tested for body weight and disease score (Disease was scored as follows; 0=no disease, 1=limp tail, 2=hind-limb paresis, 3=hind-limb paralysis, 4=hind/forelimb paralysis, and 5=death). After 2 weeks of treatment, the animal study was terminated and the remaining animals were sacrificed. As demonstrated in FIG. 10, Etomoxir treatment exerted a therapeutic effects in the mouse EAE model. AT the end of the study 52% of the treated-group was symptom-free, which was significantly different from the placebo group (p=0.009) (FIG. 10).

To test the therapeutic efficacy for the treatment of EAE in vivo, we also tested the treatment with Etomoxir in the rat EAE model. In these studies, EAE was induced in

Lewis rats with the injection with MBP protein in CFA on day 0. Due to the disease severity of this animal model, the treatment of the animals with Etomoxir or placebo started at day 7 after induction.

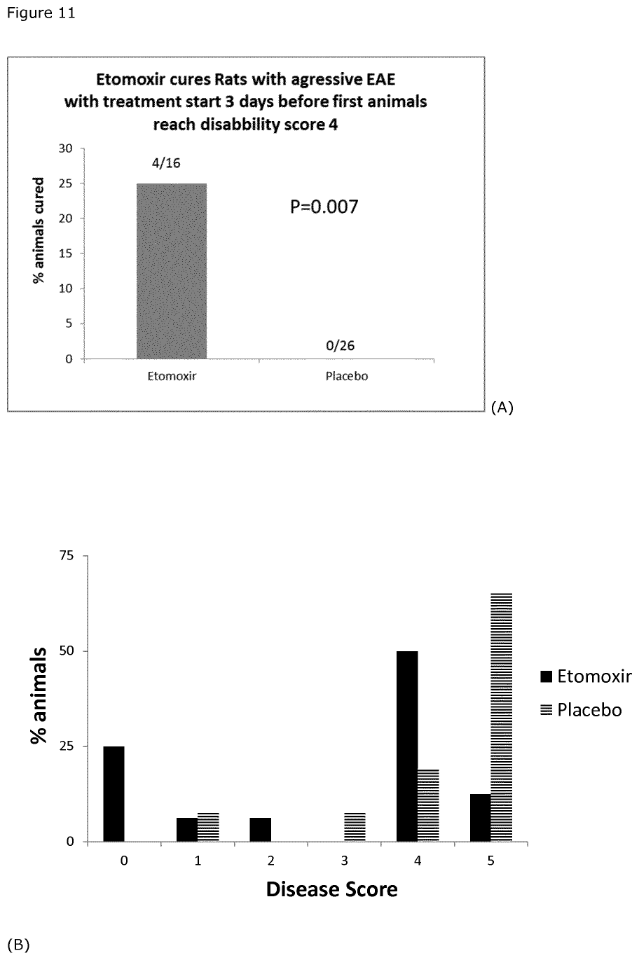

At day 11 in this model (FIG. 11a), 25% of the rats were disease- and symptom-free which was highly significant In the etomoxir-treated group (p=0.001). (11b disease score distribution at end of study)

CPT1 Blockers Produce Better Therapeutic Effects than IFNβ in Rat Models with EAE

To suggest the use of Etomoxir as the first line treatment in humans, we compared the effects of Etomoxir in rat the model with EAE, to that of interferon beta (IFNβ). Since it has been shown that IFNβ can have clinical efficacy in the rat EAE model when given early, we compared these two drugs when given at day 1 or day 5 after induction of disease. As a control, a placebo-treated group was added to the study, which was treated at day 1.

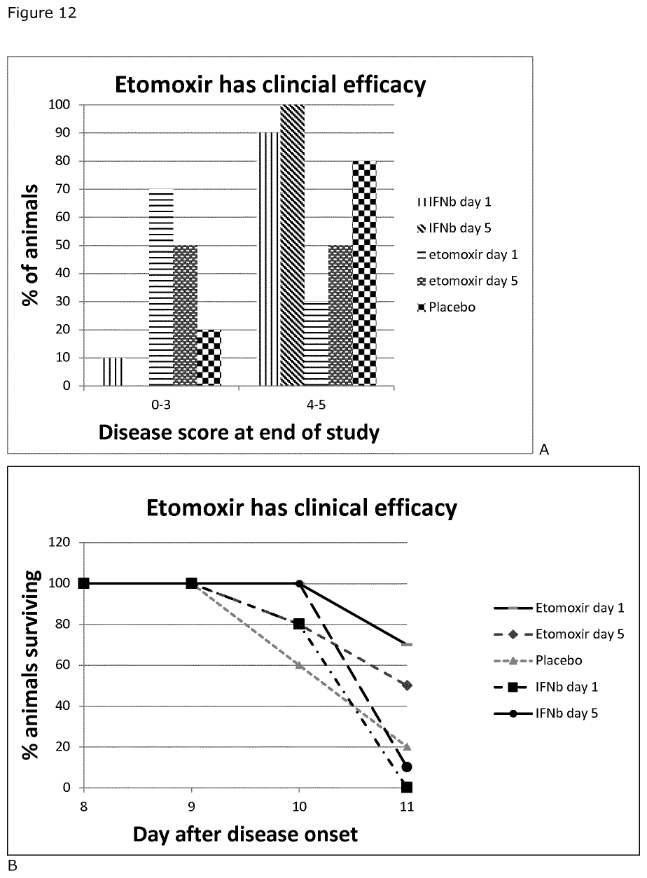

As can be seen in FIG. 12 this model produced very severe outcomes, as at day 11, most of the placebo- and IFNβ-treated rats were sacrificed due to high disease scores. IFNβ treatment did not produce therapeutic effects when given at day 1 or day 5, which were not different from placebo-treated group. In contrast, Etomoxir treatment demonstrated significantly better therapeutic effects when it was started at day 1 or day 5 (FIG. 12).

These data showed that blocking CPT1 which specifically blocks lipid metabolism produced therapeutic effects in animal models of MS. In addition Etomoxir produced better effects than IFNβ, which was known as the standard therapy for MS.

Mutations that inhibits CPT1a function in human protects the development of MS There are 2 mutations identified in humans that are present in a substantial number of people. One which has been published ((Prasad et al., 2001)) with a prevalence in the Hutterite people ((Bennett, Boriack, Narayan, Rutledge, & Raff, 2004)) the other ((Bennett et al., 2004; Rajakumar et al., 2009)(Clemente et al., 2014)) with a prevalence in Inuit people (up to 96%). In the Hutterite people, the prevalence of MS is significantly lower, compared to the people living in the same communities from 1/350 to 1/1100. The other mutation which was found is present in the Inuit population specifically the Inuit population living in the Nunavut region. These mutations seem to give a protection against getting MS, where in the normal Canadian population the prevalence is 1/350. Only 1 person with MS has been found in this Inuit population. (p<0.00000001).

### Example 8

### Effect of Etomoxir on Short Term Memory

### Study Design

Rats are put in a compartment with 3 corridors. While looking for food, they will first go in 1 corridor, then when no food is found go in the next and when they have a good short term memory, go in the 3rd after that. (this is measured as alternation ratio) MK801 destroys short term memory. Etomoxir restores short term memory. In the rats that responded memory was perfect The test of R(+)-etomoxir ethyl ester with or without MK801 in this setup shows whether R(+)-etomoxir ethyl ester can restore short term memory, and serve as a model for the effect on MS or MS associated diseases.

Etomoxir was given by sub cutaneous injection 1 hour before the test. This shows that Etomoxir can cross the blood brain barrier within 1 hour and exert its function.

### Results

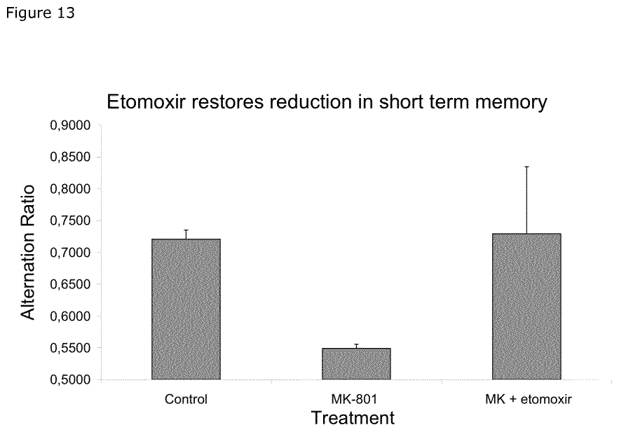

FIG. 13 shows that R(+)-etomoxir ethyl ester restores the animal's short term memory, and confirms that R(+)-etomoxir ethyl ester is effective in the treatment of amelioration on MS or MS associated diseases.

1. A method of treating or ameliorating Multiple Sclerosis (MS) or an MS associated disease comprising administering an R(+)-etomoxir ethyl ester with the chemical formula R(+)-2-[6-(4-chlorophenoxy)hexyl]-oxirane-2-carboxylic acid ethyl ester to a person in need thereof at an amount of 80 mg/day.

2-10. (canceled)

11. The method according to claim 1, wherein the 80 mg/day R(+)-etomoxir ethyl ester is administered to said person at two times of 40 mg/each.

12. The method according to claim 1, wherein the MS associated disease is selected from the group consisting of Relapsing Remitting MS (RRMS), Secondary Progressive MS (SPMS), Primary Progressive MS (PPMS), Optic Neuritis (ON), Clinically Isolated Syndrome (CIS), Amyotrofic lateral sclerose (ALS), Neuromyelitis optica (NMO), depression, and Acute Optic Neuritis (AON).

13. The method according to claim 1, wherein said R(+)-etomoxir ethyl ester is formulated in a pharmaceutical composition.

14. The method of claim 1, wherein said R(+)-etomoxir ethyl ester is formulated in a pharmaceutically effective amount.

15. The method of claim 1, wherein said R(+)-etomoxir ethyl ester is formulated for oral administration.

16. The method of claim 15, wherein said R(+)-etomoxir ethyl ester is formulated in a tablet or capsule.

17. The method of claim 1, wherein the MS associated disease is Secondary Progressive MS (SPMS).

18. The method of claim 1, wherein the treatment or amelioration is measured as a statistically significant change in Normalized Brain Volume (NBV) over a period of 6 months compared to placebo.

19. The method of claim 1, wherein the treatment or amelioration downregulates one or more of IL17a, TNFa, or IFNg.
